SEQUENCE LISTING (2 sequences)

SEQ ID NO: 1 - DNA, 24 bp, Artificial Sequence
  Oligonucleotide
taacacaactggtgtgctcctgga

SEQ ID NO: 2 - DNA, 42 bp, Artificial Sequence
  Oligonucleotide
gagctaggacctcttctgtccaggagcacaccagttgtgtta

(19) United States
(12) Patent Application Publication
(10) Pub. No.: US 2004/0038427 A1
(43) Pub. Date: Feb. 26, 2004

(54) Method for detecting and/or quantifying first molecules

(76) Inventors: Hans Kosak, Bonn (DE); Jurgen Krause, Erlangen (DE)

Correspondence Address: Fish & Richardson, 60 South Sixth Street, Suite 3300, Minneapolis, MN, 55402, US

(21) Appl. No.: 10/240,205

(22) PCT Filed: Mar. 17, 2001
(86) PCT No.: PCT/DE01/01078

(30) Foreign Application Priority Data
Mar. 29, 2000 (DE) 100 15 448.4

(51) Int. Cl. G01N 33/543

(52) U.S. Cl. 436/518; 435/6

(57) ABSTRACT

The invention relates to a method for detecting and/or quantifying the binding of first molecules (16) to second molecules (18) affine therein. The inventive method comprises the following steps: a) providing a container (10), wherein the second molecules (18) are immobilized on a wall, b) contacting the second molecules (18) to a solution containing the first molecules (16), c) incubating the container (10) in such a way that the first molecules (16) bind to the second molecules (18) and build up on the wall and d) detecting the changes in concentration of the first molecules (16) in the solution by means of radiation without removing the solution from the container (10), whereby the solution is contained in said container (10).

The invention relates to methods for detecting and/or quantifying the binding of first molecules to second molecules with affinity therefor. It further relates to methods for detecting and/or quantifying the enzymic or chemical activity of first molecules to second or third molecules with affinity therefor. Moreover, the invention relates to a microtiter plate for carrying out the method and to a use.

Methods having the following steps are known:

A) contacting first molecules having a labeling substance with immobilized second molecules with affinity therefor in a single binding reaction mixture,

B) incubating said binding reaction mixture,

C) detecting a property of said labeling substance on those first molecules which are bound to second molecules.

For methods with high throughput screening it is advantageous to carry out step C without removing unbound first molecules from the binding reaction mixture. The following methods without removal of the first molecules are known:

Detecting a fluorescence polarization. The labeling substance is a fluorophor. The change in fluorescence polarization of the labeling substance due to the binding is measured.

Detecting a change in fluorescence due to energy transfer. The labeling substance is either a fluorescence donor or a fluorescence acceptor. The binding reduces the distance between the first molecules and the second molecules. This makes possible an energy transfer between the labeling substance and a further fluorescence acceptor or fluorescence donor situated in the proximity of the second molecules.

Detecting a change in the dwell time of the first molecules in a predetermined volume. In this method which is also known as fluorescence correlation spectroscopy the labeling substance is a fluorophor. Binding to the second molecules reduces the rate of molecular motion of the first molecules. The dwell time of the fluorophor in the predetermined volume is increased.

In addition, there are test systems in which binding of unlabeled first molecules to second molecules is detected indirectly:

The second molecules are immobilized to microparticles. Fluorescent third molecules are specifically bound to the second molecules. Binding of the first molecules to the second molecules displaces the bound third molecules from the binding sites. This causes a change in the fluorescence of the microparticles, which is detected.

The second molecules are immobilized on a vessel wall which contains a scintillator. Third molecules which have a radioactive labeling substance are specifically bound to the second molecules. The labeling substance causes a scintillation which can be measured. Binding of the first molecules to the second molecules displaces the bound third molecules from the binding sites. This causes a change in scintillation, which is measured.

The known homogeneous test systems are technically complicated and partly require harmful substances.

WO 95/18376 describes a method for determining the concentration of an analyte in a liquid. This involves contacting the liquid with a probe which is immobilized on a solid phase and which specifically binds said analyte. This is followed by determining the proportions of occupied and unoccupied binding sites of the probe by means of back titration. The result is compared with a previously determined standard—the method used requires many steps. It is time-consuming and susceptible to contaminations.

U.S. Pat. No. 6,020,207 discloses a method for optical detection of chemical compounds. This involves immobilizing a probe on the inner wall of a capillary. A solution containing the analyte is introduced into the capillary. The chemical compound is detected by measuring light absorption on the inner wall of the capillary, which is changed due to binding of the chemical compound to the probe.—The measurement is complicated and requires extensive equipment.

WO 99/44065 describes a method for detecting an analyte in a sample. This involves pumping the solution containing said analyte through a capillary on whose inner wall a probe has been immobilized. Compounds which contain a label detectable by means of modified Raman spectroscopy are bound to the probe. If the analyte is present, said compounds are released and can be detected by means of modified Raman spectroscopy.

DE 44 07 142 A1 relates to a heterologous immunoassay in which a probe is immobilized on a solid support, for example a flow-through cuvette. An analyte contained in a solution is bound to the probe, then detached again and detected, for example by means of fluorescence or UV absorption.

DE 198 28 837 A1 describes a method in which a probe is immobilized on a ring. To detect an analyte bound to the probe said ring may be put into a reaction chamber of a UV analyzer. The proposed detection method is complicated.

It is an object of the present invention to remove the disadvantages of the prior art. In particular, methods, a microtiter plate and a use are to be provided, which make it possible to achieve a high sample throughput in a simple and inexpensive manner. After contacting the second molecules with the first molecules, no further pipetting or washing step should be required.

This object is achieved by the features of claims 1 to 5, 34 and 40. Expedient embodiments result from the features of claims 5 to 33, 35 to 39 and 41 and 42.

The method of the invention is suitable for detecting and/or quantifying the binding of first molecules to second molecules with affinity therefor. It is likewise suitable for quantifying the enzymic or chemical activity of first molecules to second or third molecules with affinity therefor. Each of the method steps a to d make it possible to carry out the method in a simple, quick and inexpensive manner.

In step d, concentration can be detected by means of electromagnetic radiation such as light or by means of particle radiation such as neutron radiation. This takes place preferably in a region of the vessel, which is at a maximum distance from the wall. This increases the accuracy of the method. The methods of the invention have the advantage that they can be carried out with little pipetting. They are easy to manage and inexpensive. They require little material and space. They make possible a high sample throughput and are well-suited for automation. This is achieved in particular by the fact that, after contacting the second molecules with a solution containing the first molecules, no further mechanical steps, in particular no separation and washing steps, are required. Due to the low number of operational steps, the risk of distorted results, in particular due to pipetting errors, is low. The method may be carried out using a large number of vessels and different first, second, third and/or fourth molecules simultaneously or in quick succession. The method is ideally suited for the search for new pharmacological active compounds.

In the case of the methods for detecting and/or quantifying the binding (=method group I) by using third or fourth molecules, it is particularly advantageous for said third or fourth molecules to be associated with the second molecules or with the wall. In this way, it is not necessary to add the third or fourth molecules to the solution or the vessel in a pipetting step.

It is furthermore advantageous, in the case of the methods according to method group I using fourth molecules, if the affinity of the fourth molecules for the second molecules is not greater or not substantially greater than the affinity of the first molecules for the second molecules. This facilitates displacement of the fourth molecules from their binding sites on the second molecules. Likewise, binding of the fourth molecules to the second molecules is prevented more readily.

In the case of the methods according to method group I using fourth molecules, it is advantageous if the number of the first molecules is greater than the number of the second molecules and the number of the second molecules is greater than the number of the fourth molecules. This causes a competition of the first molecules and the fourth molecules for the binding sites on the second molecules. The concentration of the fourth molecules is greatly influenced by the concentration of the first molecules and the affinity thereof for the second molecules.

In the case of a method of the method group I without fourth molecules, preference is given to the number of the second molecules being greater than the number of the first molecules. This results in a rapid and distinct change in concentration of the first molecules or of the first and third molecules in the solution contained in the vessel. In the case of a method using third molecules, it is advantageous if the number of the first molecules is not less than the number of the third molecules. This prevents third molecules from remaining in the solution after binding of the first molecules to the second molecules. The third molecules remaining in the solution cause a background signal in step d.

In the case of the method for detecting and/or quantifying the enzymic or chemical activity of first molecules (=method group II), the first molecule may be a cleaving enzyme, preferably a protease, a peptidase, nuclease, helicase or lipase, or a sugar-degrading enzyme. The second molecule may form a substrate for the first molecule, which is preferably derived from a protein, peptide, a nucleic acid, helicase or a monomeric or polymeric sugar. It is further possible that the third molecule is a cleaving enzyme, preferably a protease, peptidase, nuclease, helicase or lipase or a sugar-degrading enzyme. Expediently, the second molecule forms a substrate for the third molecule, which is preferably derived from a protein, peptide, a nucleic acid, helicase or a monomeric or polymeric sugar. The first molecule may be an agonist, an antagonist or a competitor with respect to the third molecule. It is furthermore expedient that binding of the second molecule to the first molecule prevents cleavage of the second molecule by the third molecule.

In an advantageous embodiment which can be applied equally to both method groups, the solution is irradiated for detection by a light beam, in particular of a defined wavelength, preferably a laser beam. The light beam may irradiate parallel to the wall. It may be polarized. A fluorescence, a diffraction, an absorption or an optical activity may be measured for detection.

Advantageously, the solution additionally contains fifth molecules which serve as internal markers and which have no specific affinity to the first, second, third or fourth molecules. The fifth molecules may be employed for detecting a change in volume and thus a change in concentration due to evaporation of water. Furthermore, the fifth molecules may serve to determine the amount of unspecific bindings. In this connection, a decrease in the concentration of the fifth molecules indicates unspecific binding to the wall and/or to the second molecules.

In a preferred embodiment, the first, third, fourth and/or fifth molecules or solution components associated therewith are fluorescent, light-diffracting, light-absorbing or optically active. Solution components associated therewith may be, for example, antibodies which can specifically bind the first, third, fourth and/or fifth molecules.

Step d is advantageously carried out by multiple or continuous determination of the concentration of the first, third or fourth molecules, while step c is carried out in particular at the start and end thereof. The concentration may be determined, for example, by comparing the optical properties of the solution with those of various standard solutions.

Free binding sites on the vessel wall are preferably saturated by sixth molecules bound thereto. The sixth molecules should have physical/chemical properties such as charge, molecule size, etc. similar to those of the second molecules. However, they should have no affinity for the first molecules. This makes it possible to suppress unspecific binding to free binding sites on the vessel wall. Any binding other than to the specific binding sites of the second molecules is unspecific.

The solution advantageously contains at least one additive inhibiting unspecific binding, in particular a detergent, a protein, a protein mixture or a salt. Said additive does not suppress specific binding of the first molecules to the specific binding sites of the second molecules. It inhibits unspecific bindings of the first, second, third, fourth and fifth molecules to one another and to the vessel wall or to the sixth molecules bound there. Examples of suitable detergents are Tween-20, Nonidet or SDS. Suitable salts are those suppressing unspecific ionic bonds. Possible examples of proteins or protein mixtures are skimmed milk, bovine serum albumin and casein.

In an advantageous embodiment, a specific change in concentration is determined by subtracting the value of a change in concentration when carrying out the method without second molecules from the value of the change in concentration according to step d. A change in concentration, which is caused only by binding of the first molecules to the second molecules, is specific.

Preferably, the second molecules are immobilized at a predetermined site of their structure. This makes it possible to prevent the second molecules from being immobilized on the site of their structure, which has affinity for the second or fourth molecules. Immobilizing of this kind would restrict the accessibility of these sites for first or fourth molecules with affinity therefor. The second molecules are advantageously bound to the wall via linkers or spacers. This improves accessibility of the second molecules. Linkers can improve or make possible the immobilizing of second molecules which can be immobilized directly only with difficulty, if at all.

The vessel preferably forms a cavity of a microtiter plate having at least 96, in particular 384, cavities. In a preferred embodiment, the vessel has essentially no immobilized second molecules at the entry and/or exit points of the light beam, preferably at the base of the vessel. In step d, this prevents first, third or fourth molecules which are bound directly or indirectly to immobilized second molecules at the entry and/or exit points of the light beam from interfering with the detection. It is particularly advantageous if the vessel is a capillary which is open at its ends. A capillary here means a tube which is open at both ends and has an inner diameter of up to 2 millimeters. The light beam can shine through the capillary without irradiating the capillary wall. It is impossible for bound first, third or fourth molecules to affect the detection. The solution is preferably introduced into the capillary by means of capillary forces. This ensures introduction of a defined volume. A pipetting step is not required. A source of error caused thereby is eliminated.

The quotient of the wall area in mm² and the solution volume in mm³ is preferably greater than 1 mm⁻¹, and preferentially greater than 3 mm⁻¹. In this case, the vessel has an elongated shape. A predetermined volume can be contacted with a large wall area containing immobilized second molecules. At the same time, a long path of the light beam through the solution is ensured. An elongated vessel geometry can increase the sensitivity of the method considerably.

The first, second, third, fourth, fifth or sixth molecules can be selected from the following group: peptides, proteins, nucleic acids, sugars, polymers, messengers, cells, cell fragments, viruses, components thereof or fragments of said components, capsids, components thereof or fragments of said components and hormones. In an advantageous embodiment of the invention, the method of the invention is carried out in a number of vessels, in particular capillaries, simultaneously or in quick succession.

A microtiter plate with a multiplicity of cavities having, in each case, a wall and a base is particularly suitable for carrying out the method of the invention, with the wall being activated for binding of the molecules and the base not being activated for the binding of second molecules. Such a microtiter plate allows the user to immobilize in each case the second molecules of interest to him on the wall himself.

The microtiter plate which is expediently made of plastic is activated according to conventional methods. Activation of the cavity base can be prevented by covering the base during activation, for example. However, it is also possible to design the base as a separate part and to attach it to the already activated walls.

According to a further design feature, second molecules are immobilized on the wall and the base has essentially no immobilized second molecules. In this connection, in each case different second molecules may be immobilized on the wall. This allows carrying out the method with a high throughput.

Advantageously, at least a section of the wall is prepared from a porous material, it being possible for the porous material to be selected from the following group: cellulose, nitrocellulose, nylon, agarose, paper or cardboard. The base is expediently prepared from a transparent or translucent material. This allows measuring the absorption of a laser beam in a transmitted-light process. According to a further design feature, third or fourth molecules are associated with the wall or the second molecules.

The invention furthermore relates to the use of capillaries on whose walls the second molecules are immobilized for carrying out the method of the invention. In this connection, the capillaries may differ, at least partially, due to different second molecules. Preferably, capillaries with different second molecules differ further, in particular in their sizes or labels. The labels may comprise further molecules or, in particular fluorescent, dyes. This makes is possible to identify capillaries on whose walls particular second molecules are immobilized. It is further possible for the third or fourth molecules to be associated with the wall.

The invention is illustrated in more detail below by the drawing and on the basis of exemplary embodiments, where

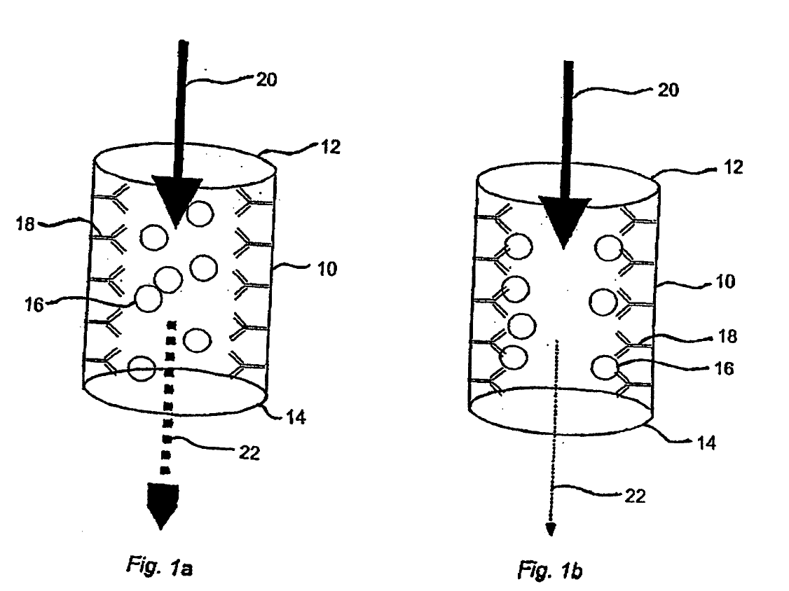

FIGS. 1a, 1b show a diagrammatic representation of irradiating a vessel containing a solution and having first molecules and immobilized second molecules,

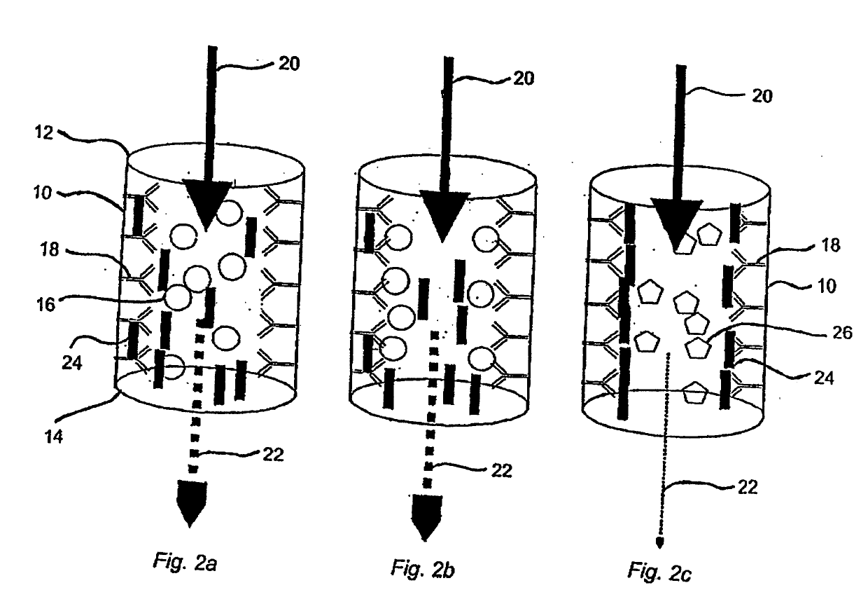

FIGS. 2a, 2b, 2c show a diagrammatic representation of irradiating a vessel containing a solution and having second, fourth and first or fifth molecules,

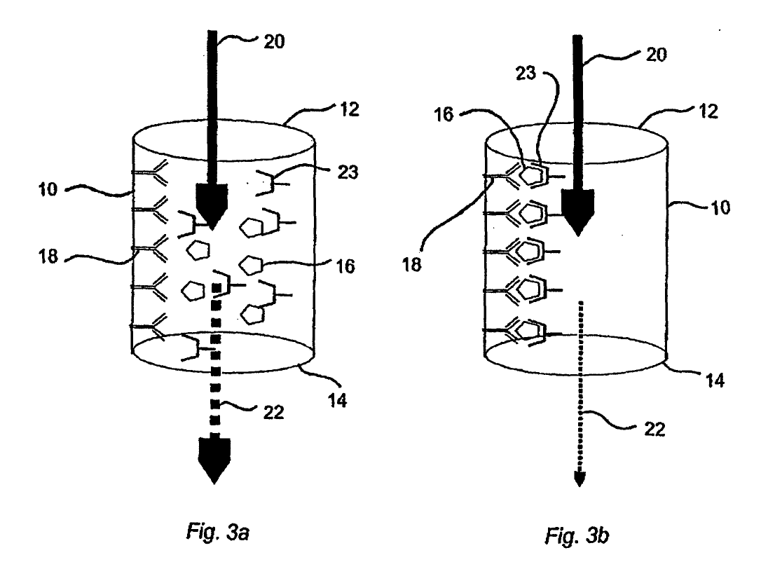

FIGS. 3a, 3b show a diagrammatic representation of irradiating a vessel containing a solution and having first, second and third molecules,

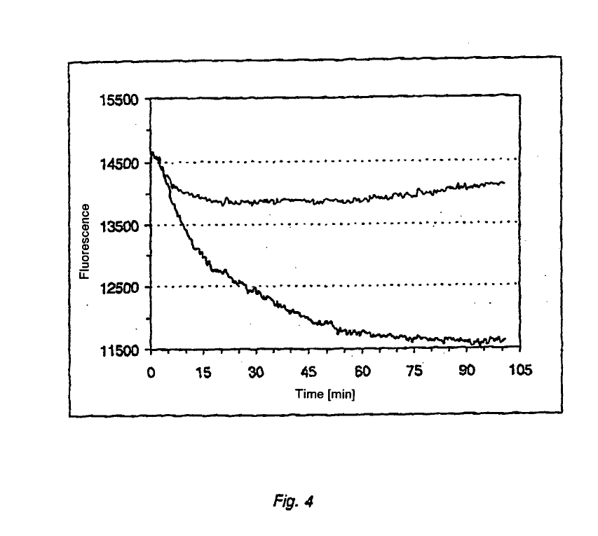

FIG. 4 shows a diagram of the time course of the fluorescence of fluorescently labeled oligonucleotides, which is detected in the wells of an 8-well strip,

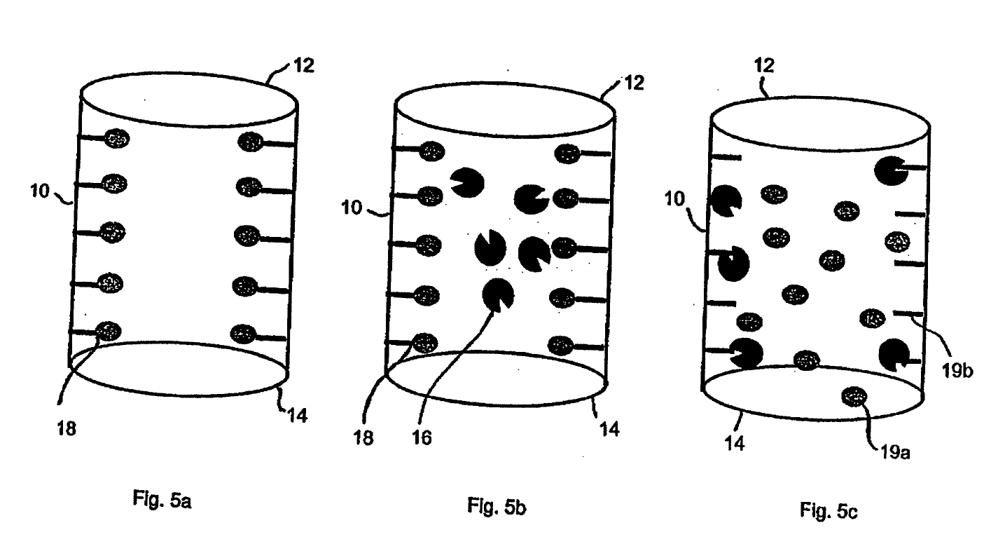

FIGS. 5a, b, c show a diagrammatic representation of a vessel containing a solution and having first molecules and immobilized second molecules, and

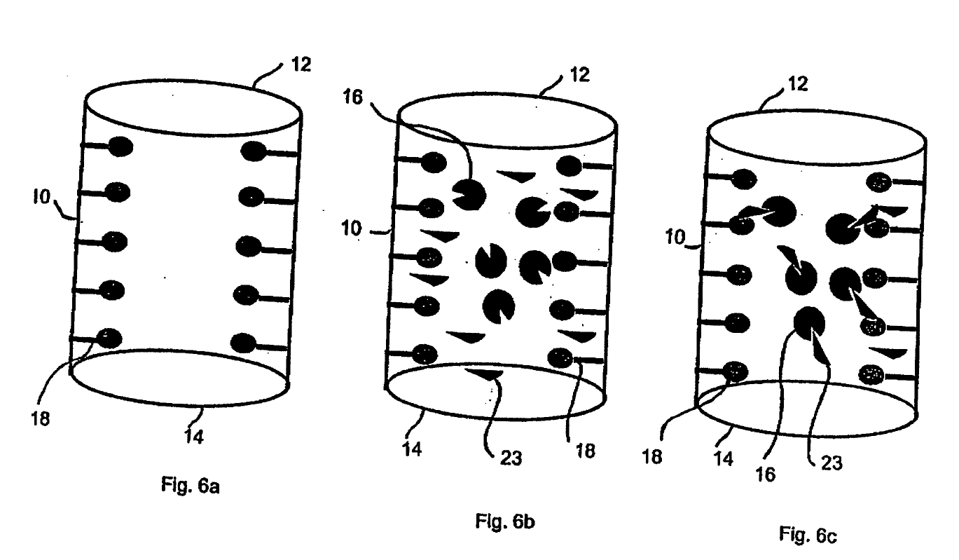

FIGS. 6a, b, c show a diagrammatic representation of a vessel containing a solution and having first molecules, immobilized second molecules and third molecules.

FIG. 1a shows a section of a vessel 10 having a first end 12 and a second end 14. The vessel 10 contains a solution which contains the first molecules 16. The second molecules 18 with affinity therefor are immobilized on the wall of the vessel 10. The light beam 20 irradiating the vessel 10 at the first end 12 is modulated by an interaction with the first molecules 16, for example by absorption. The light beam 22 exiting at the second end 14 has altered properties compared with the irradiating light beam 20. The change in these properties is a measure of the concentration of the first molecules 16 in the solution. FIG. 1b shows the vessel 10 depicted in FIG. 1a after incubating. The first molecules 16 are bound to the second molecules 18. The light beam 20 irradiating the vessel 10 is not modulated by interactions with the first molecules 16. In the case of light-absorbing first molecules 16, the exiting light beam 22 is attenuated only by the absorption of light by the solution but not, as in FIG. 1a, by the absorption of light by the first molecules 16.

FIG. 2a depicts a section of a vessel 10 containing second molecules 18 bound thereto. The solution contained in the vessel 10 contains first molecules 16 and fourth molecules 24 both of which have an affinity for the second molecules 18. The irradiating light beam 20 is modulated here by interaction with the fourth molecules 24 but not with the first molecules 16. The interaction may be caused by the fact that the fourth molecules 24 are light-absorbing. The exiting light beam 22 has altered properties compared with the irradiating light beam 20. FIG. 2b shows the situation after incubating. The first molecules 16 are bound to the second molecules 18. They prevent the fourth molecules 24 from binding to the second molecules 18. The concentration of the fourth molecules 24 in the solution is unchanged. The properties of the exiting light beam 22 have not changed compared with the situation depicted in FIG. 2a. This indicates binding of the first molecules 16 to the second molecules 18. FIG. 2c shows the situation after incubating, if the solution previously contained, instead of the first molecules 16, fifth molecules 26 which have no affinity for the second molecules 18. The fourth molecules 24 then bind to the second molecules 18 during incubating. After incubating, the irradiating light beam 20 is not modulated by an interaction with the fourth molecules 24. In the case of light-absorbing fourth molecules 24, the intensity of the exiting light beam 22 is higher than at the start of the incubation.

FIG. 3a shows a section of a vessel 10. The vessel 10 contains a solution containing first molecules 16 and third molecules 23 with affinity therefor. Second molecules 18 are immobilized on the wall of the vessel 10. They have an affinity for the first molecules 16. The light beam 20 irradiating the vessel 10 at the first end 12 is modulated by an interaction with the third molecules 23, for example by absorption. The exiting light beam 22 has altered properties compared with the irradiating light beam 20. FIG. 3b shows the situation after incubating. The first molecules 16 are bound to the second molecules 18 and the third molecules 23. The binding sites of the second 18 and third molecules 23 on the first molecules 16 are not identical. The third molecules 23 are removed from the solution. The light beam 20 irradiating the vessel 10 is no longer modulated by interactions with the third molecules 23.

FIG. 4 shows the time course of the fluorescence of fluorescently labeled oligonucleotides I, which is detected in Streptavidin-coated wells of an 8-well strip (Boehringer Mannheim, No. 1664778). A well I of the 8-well strip has been coated with a 5′-terminally biotinylated oligonucleotide II of the sequence 5′-TAA CAC AAC TGG TGT GCT CCT GGA-3′ according to sequence listing No. 1. For this purpose, 300 μl of a buffer solution I containing 167 nM oligonucleotide II have been introduced into the well I. The buffer solution I consists of an aqueous solution of 10 mM TrisCL [sic] and 1 mM EDTA, pH 8.0. 300 μl of buffer solution I only have been introduced into a well II of the 8-well strip. After incubation at 37° C. for one hour, the buffer solutions I have been removed by suction from the wells I and II. The wells I and II have been washed 5 times with 320 μl of the buffer solution I. Subsequently, 300 μl of a buffer solution II containing 167 mM oligonucleotide I of the sequence 5′-GAG CTA GGA CCT CTT CTG TCC AGG AGC ACA CCA GTT GTG TTA-3′ according to sequence listing No. 2, fluorescently labeled with 5[6]-carboxytetramethylrhodamine at the 5′ terminus, have been introduced in each case into the wells I and II. The buffer solution II consists of an aqueous solution of 150 mM NaCl, 10 mM TrisCl, 1 mM EDTA and 0.2% Tween-20, pH 8.0. The fluorescence in the wells I and II has been measured at 590 nm with excitation at 540 nm. Measuring was carried out in 25-second intervals at room temperature by means of a microtiter-plate spectrophotometer. FIG. 4 depicts the fluorescence in arbitrary units. The thicker line indicates the fluorescence measured in well I and the thinner line the fluorescence measured in well II. The decrease in fluorescence in well II indicates unspecific binding of the oligonucleotide I to the wall of well II. The difference between the decrease in fluorescence in well II and the decrease in fluorescence in well I indicates specific binding of the oligonucleotide I to the oligonucleotide II.

FIGS. 5a to c show diagrammatically the function of a method for detecting and/or quantifying the catalytic activity of first molecules to second molecules with affinity therefor. The first molecules 16 used here are enzymes. Second molecules 18 are bound to the wall, but not to the base, of the vessel 10. In the present case, said second molecules may be peptides, nucleic acids, sugars, lipids and similar substances which constitute a substrate for the enzyme. Advantageously, the second molecules 18 are labeled with a fluorophore. The light path of the light beam irradiating the vessel 10 (not shown here) runs parallel to the walls. It does not reach the area of the second molecules 18.

FIG. 5b shows the vessel 10 after adding the first molecules 16 to be detected. Examples of first molecules 16 which may be used here are enzymes such as proteases, nucleases, sugar-degrading enzymes, lipases or other enzymes. The first molecules may also be parts of larger enzyme complexes or cells. Furthermore, the first molecules may be produced and/or secreted by cells.

FIG. 5c shows the vessel 10 after incubating. Fragments 19a have been cleaved off the second molecules 18 due to the activity of the enzymes used as first molecules 16 here by way of example. Residues 19b remain on the wall of the vessel 10, while the fragments 19a formed due to the activity of the first molecules 16 are distributed evenly throughout the volume of the vessel 10. They absorb light of the irradiating light beam (not shown here). Absorption of the light can detect the activity of the first molecules 16.

FIGS. 6a to c show diagrammatically the course of the method when adding an enzyme as first molecule 16 and using a competitor as third molecule 23. In a vessel 10, second molecules 18 are immobilized on the walls but not on the base. Examples of second molecules 18 may be peptides, nucleic acids, sugars, lipids or other substances which constitute a substrate for the enzyme. Advantageously, the second molecules 18 are labeled, for example with a fluorophore. A light beam (not shown here) penetrates the vessel 10 parallel to the walls thereof, light not being applied to the area of the second molecules 18. As an alternative to the light beam, it is also possible to observe fluorescence in a central area of the vessel 10.

FIG. 6b shows the vessel 10 after addition of the first molecules 16. Third molecules 23 are present in the vessel. In the present example, the first molecules 16 used are competitors and the third molecules 23 used are proteases. However, suitable third molecules 23 are also nucleases, sugar-degrading enzymes, lipases or other enzymes. Suitable first molecules 16 are also inhibitors, enhancers, inductors and the like.

FIG. 6c shows the vessel 10 after incubating the first molecules 16 to be detected with the third molecules 23 and the immobilized second molecules 18. Due to the activity of the competitors used as first molecules 16, the activity of the third molecules 23, in this case proteases, have been specifically altered. If the first molecules 16 are inhibitors of the third molecules 23, degradation of the second molecules 18 is specifically inhibited. The inhibition can be detected on the basis of the fragments 19a formed in the vessel 10. A reduced increase in fragments 19a formed in comparison with a control with no first molecules 16 added indicates the presence of the first molecules 16 in the vessel 10.

List of Reference Numbers

10 Vessel,

12 first end,

14 second end,

16 first molecules,

18 second molecules,

19a fragment,

19b residue,

20 irradiating light beam,

22 exiting light beam,

23 third molecules,

24 fourth molecules,

26 fifth molecules

1. A method for detecting and/or quantifying the binding of first molecules (16) to second molecules (18) with affinity therefor, comprising the following steps:
a) providing a vessel (10) in which the second molecules (18) are immobilized on a wall,
b) contacting the second molecules (18) with a solution containing the first molecules (16),
c) incubating the vessel (10) so that the first molecules (16) bind to the second molecules (18) and accumulate on the wall and
d) detecting the changes in concentration of the first molecules (16) in the solution contained in the vessel (10) by means of radiation, without removing solution from the vessel.

2. A method for detecting and/or quantifying the binding of first molecules (16) to second molecules (18) with affinity therefor, comprising the following steps:
a) providing a vessel (10) in which the second molecules (18) are immobilized on a wall,
b) contacting the second molecules (18) with a solution containing the first molecules (16), with third molecules (23) having an affinity for the first molecules (16) being contained in the solution or in the vessel (10),
c) incubating the vessel (10) so that the first molecules (16) bind to the second (18) and third molecules (23) and accumulate on the wall and
d) detecting the changes in concentration of the third molecules (23) in the solution contained in the vessel (10) by means of radiation, without removing solution from the vessel.

3. A method for detecting and/or quantifying the binding of first molecules (16) to second molecules (18) with affinity therefor, comprising the following steps:
a) providing a vessel (10) in which the second molecules (18) are immobilized on a wall,
b) contacting the second molecules (18) with a solution containing the first molecules (16), with fourth molecules (24) having an affinity for the second molecules (18) being contained in the solution or in the vessel (10),
c) incubating the vessel (10) so that the first molecules (16) bind to the second molecules (18) and the fourth molecules (24) are, at least partially, dissolved or remain and
d) detecting the changes in concentration of the fourth molecules (24) in the solution contained in the vessel (10) by means of radiation, without removing solution from the vessel.

4. A method for detecting and/or quantifying the enzymic or chemical activity of first molecules (16) toward second molecules (18) with affinity therefor, comprising the following steps:
a) providing a vessel (10) in which the second molecules (18) are immobilized on a wall,
b) contacting the second molecules (18) with a solution containing the first molecules (16),
c) incubating the vessel (10) so that the first molecules (16) convert the second molecules (18) with release of a fragment (19a) of the second molecules (18) and
d) detecting the change in concentration of the fragment (19a) in the solution contained in the vessel (10) by means of radiation, without removing solution from the vessel.

5. A method for detecting and/or quantifying the enzymic or chemical activity of first molecules (16) toward second (18) or third molecules with affinity therefor, comprising the following steps:
a) providing a vessel (10) in which the second molecules (18) are immobilized on a wall,
b) contacting the second molecules (18) with a solution containing the first molecules (16), with third molecules having an affinity for the second molecules being contained in the solution,
c) incubating the vessel (10) so that the first molecules (16) bind to the second (18) or third molecules, whereby a conversion causing the release of a fragment (19a) of the second molecules (18) is suppressed or increased by the third molecules, and
d) detecting the change in concentration of the fragment (19a) in the solution contained in the vessel (10) by means of radiation, without removing solution from the vessel (10).

6. The method as claimed in claim 2 or 3, in which the third (23) or fourth molecules (24) contained in the vessel (10) are associated with the second molecules (18) or the wall.

7. The method as claimed in claim 3, in which the affinity of the fourth molecules (24) for the second molecules (18) is not greater or not substantially greater than the affinity of the first molecules (16) for the second molecules (18).

8. The method as claimed in claim 3, in which the number of the first molecules (16) is greater than the number of the second molecules (18) and the number of the second molecules (18) is greater than the number of the fourth molecules (24).

9. The method as claimed in claim 1 or 2, in which the number of the second molecules (18) is greater than the number of the first molecules (16).

10. The method as claimed in claim 2, in which the number of the first molecules (16) is not less than the number of the third molecules (23).

11. The method as claimed in claim 4, in which the first molecule (16) is a cleaving enzyme, preferably a protease, peptidase, nuclease, helicase or lipase, or a sugar-degrading enzyme.

12. The method as claimed in claim 4 or 11, in which the second molecule (18) forms a substrate for the first molecule (16), which is preferably derived from a protein, peptide, a nucleic acid, helicase or a monomeric or polymeric sugar.

13. The method as claimed in claim 5, in which the third molecule is a cleaving enzyme, preferably a protease, peptidase, nuclease, helicase or lipase, or a sugar-degrading enzyme.

14. The method as claimed in claim 5, in which the second molecule (18) forms a substrate for the third molecule, which is preferably derived from a protein, peptide, a nucleic acid, helicase or a monomeric or polymeric sugar.

15. The method as claimed in claim 5, in which the first molecule (16) is an agonist, an antagonist or a competitor with respect to the third molecule.

16. The method as claimed in claim 5, in which binding of the second molecule to the first molecule prevents cleavage of the second molecule by the third molecule.

17. The method as claimed in any of the preceding claims, in which a light beam (20), in particular of a defined wavelength, preferably a laser beam, is used to irradiate the solution for detection.

18. The method as claimed in claim 17, in which the light beam (20) is introduced parallel to the wall.

19. The method as claimed in claim 17 or 18, in which the light beam (20) is polarized.

20. The method as claimed in any of the preceding claims, in which a fluorescence, a diffraction, an absorption or an optical activity is measured for detection.

21. The method as claimed in any of the preceding claims, in which the solution additionally contains fifth molecules (26) which serve as internal markers and have no specific affinity for the first (16), second (18), third (23) or fourth molecules (24).

22. The method as claimed in any of the preceding claims, in which the first (16), third (23), fourth (24) and/or fifth molecules (26) or fragments of these molecules or components associated therewith are fluorescent, light-diffracting, light-absorbing or optically active.

23. The method as claimed in any of the preceding claims, in which step d is carried out by multiple or continuous determination of the concentration of the first (16), third (23) or fourth molecules (24), while carrying out step c, in particular at the start and end thereof.

24. The method as claimed in any of the preceding claims, in which free binding sites on the wall of the vessel (10) are saturated by sixth molecules bound thereto.

25. The method as claimed in any of the preceding claims, in which the solution contains at least one additive inhibiting unspecific binding, in particular a detergent, a protein, a protein mixture or a salt.

26. The method as claimed in any of the preceding claims, in which a specific change in concentration is determined by subtracting the value of a concentration change when carrying out the method without second molecules from the value of the concentration change according to step d.

27. The method as claimed in any of the preceding claims, in which the vessel (10) has the form of a cavity of a microtiter plate containing at least 96, in particular 384, cavities.

28. The method as claimed in any of the preceding claims, in which the vessel (10) has essentially no immobilized second molecules (18) at the point of entry and/or exit of the light beam (20, 22), preferably at the base of the vessel (10).

29. The method as claimed in any of the preceding claims, in which the vessel (10) is a capillary open at its ends (12, 14).

30. The method as claimed in claim 29, in which the capillary is filled with the solution by means of capillary forces.

31. The method as claimed in any of the preceding claims, in which the quotient of the wall area in mm² and the solution volume in mm³ is greater than 1 mm⁻¹, preferably greater than 3 mm⁻¹.

32. The method as claimed in any of the preceding claims, in which the first (16), second (18), third (23), fourth (24), fifth (26) or sixth molecules are selected from the following group: peptides, proteins, nucleic acids, sugars, polymers, messengers, cells, cell fragments, viruses, components thereof or fragments of said components, capsids, components thereof or fragments of said components and hormones.

33. The method as claimed in any of the preceding claims, in which the method is carried out simultaneously or in quick succession in a number of vessels (10), in particular capillaries.

34. A microtiter plate for carrying out a method as claimed in any of claims 1-33, having a multiplicity of cavities which have in each case a wall and a base, said wall being activated for the binding of second molecules (18),
characterized in that
the base is not activated for the binding of second molecules (18).

35. The microtiter plate as claimed in claim 34, in which second molecules (18) are immobilized on the wall and the base has essentially no immobilized second molecules (18).

36. The microtiter plate as claimed in claim 35 or 36, in which at least sections of the wall are made of a porous material.

37. The microtiter plate as claimed in claim 37, in which the porous material is selected from the following group: cellulose, nitrocellulose, nylon, agarose, paper, cardboard.

38. The microtiter plate as claimed in any of claims 35 to 38, in which the base is made of a transparent or translucent material.

39. The microtiter plate as claimed in claims 35 to 39, in which third (23) or fourth molecules (24) are associated with the wall or the second molecules (18).

40. The use of capillaries on whose walls second molecules (18) are immobilized for carrying out the method as claimed in any of claims 1-33.

41. The use as claimed in claim 41, in which the capillaries have a label indicating the type of the second molecules (18).

42. The use as claimed in claim 41 or 42, in which the third (23) or fourth molecules (24) are associated with the wall or the second molecules (18).
